(19) United States
(12) Patent Application Publication
(10) Pub. No.: US 2022/0047606 A1
(43) Pub. Date: Feb. 17, 2022

(54) TREATMENT METHOD WITH IAP INHIBITOR

(71) Applicants: Ascentage Pharma (Suzhou) Co., Ltd., Suzhou (CN); Ascentage Pharma Group Corp Limited, Hong Kong (HK)

(72) Inventors: Yifan Zhai, Suzhou (CN); Xiaoyong Zhang, Suzhou (CN); Jinlin Hou, Suzhou (CN); Dajun Yang, Suzhou (CN)

(21) Appl. No.: 17/297,428

(22) PCT Filed: Nov. 19, 2020
(86) PCT No.: PCT/CN2020/130068

§ 371 Date: May 26, 2021

(30) Foreign Application Priority Data
Nov. 19, 2019 (CN) PCT/CN2019/119437
Feb. 4, 2020 (CN) PCT/CN2020/074263

(51) Int. Cl. A61K 31/551; A61K 31/708; A61K 9/00; A61P 31/14

(52) U.S. Cl. CPC A61K 31/551; A61P 31/14; A61K 9/0019; A61K 31/708

(57) ABSTRACT

Improved methods for treating hepatitis B (HB) in patients with (5S,5′S,8S,8′S,10aR,10a′R)-3,3′-(1,3-phenylenedisulfonyl)bis(N-benzhydryl-5-((S)-2-(methylamino)propanamido)-6-oxodecahydropyrrolo[1,2-a][1,5]diazocine-8-carboxamide) (Compound (I)) are disclosed herein. Dosing regimens of Compound (I) for the treatment of HBV infection are provided.

### CROSS-REFERENCE TO RELATED APPLICATIONS

The present application claims the benefits of International Application No. PCT/CN2019/119437 filed on Nov. 19, 2019 and International Application No. PCT/CN2020/074263 filed on Feb. 04, 2020. The contents of the above International Applications are incorporated herein by reference in their entireties.

### FIELD OF THE INVENTION

The present invention relates to a method of treating hepatitis B virus (HBV). The method involves the administration of an Inhibitor of Apoptosis (IAP) antagonist (e.g., compound (I) disclosed herein).

### BACKGROUND OF THE INVENTION

Hepatitis B is a common disease with a worldwide distribution, with an estimated 280,000,000 as being carriers of HBV. Globally, HBV infection is most common in the developing countries of Southeast Asia, Africa and parts of South America, where vertical transmission to infants at an early age results in a high proportion of infected individuals becoming chronic carriers of HBV. Males acquiring HBV as infants have approximately a 40% chance of dying from cirrhosis or primary hepatocellular carcinoma as a result of chronic HBV infection. In contrast, females infected at birth have about a 15% chance of dying a similar death from chronic hepatitis B infection.

Hepatitis B infection remains difficult to treat despite several drugs now in clinical use, including interferon α2b (IFN α2b), IFN α2a, lamivudine, adefovir and entecavir. Treatment is either ineffective at the outset, or can become so by the emergence of drug resistant viruses. Existing drug regimens have also been known to suffer from being long-term, expensive and associated with undesirable side effects. For example, while lamivudine has been applied with some success in the treatment of HBV infection, it is associated with an increasing risk of resistance, which can be as high as 45-55% after the second year of treatment. Moreover, HBV cannot be completely eliminated from the liver under such therapy, so that reactivation of a HBV infection occurs in many cases even after cessation a treatment. When end-stage liver failure occurs in patients with chronic HBV infection, liver transplantation is the only alternative form of treatment. However, as HBV infection persists, the graft can become infected, thus limiting patient and graft survival.

The present invention is concerned with the development of a novel approach to the treatment of HBV.

### SUMMARY OF THE INVENTION

Disclosed herein are improved methods for treating hepatitis B virus (HBV) in patients with (5S ,5′S ,8S,8′S,10aR,10a′R)-3,3′-(1,3-phenylenedisulfonyl)bis(N-benzhydryl-5-((S)-2-(methylamino)propanamido)-6-oxodecahydropyrrolo [1,2-a][1,5]diazocine-8-carboxamide) (Compound (I)). The disclosure provides dosing regimens of Compound (I) for the treatment of HBV.

In another aspect, the present disclosure provides a method of treating hepatitis B, comprising administering to a subject in need thereof: a) an amount of 7 mg to 45 mg of Compound (I), or a pharmaceutically acceptable salt thereof; and b) an amount of 0.2 mg to 1 mg of entecavir.

In another aspect, the present disclosure provides use of Compound (I) (in an amount of 7 mg to 45 mg), or a pharmaceutically acceptable salt thereof in the preparation of a medicament for use in combination with entecavir (in an amount of 0.2 mg to 1 mg) to treat hepatitis B (e.g. chronic hepatitis B).

In another aspect, the present disclosure provides Compound (I), or a pharmaceutically acceptable salt thereof for use in combination with entecavir to treat hepatitis B (e.g. chronic hepatitis B).

### BRIEF DESCRIPTION OF THE DRAWING

FIG. 1 shows Mean HBsAg decline from baseline to the end of follow-up in 4 dose-escalating cohorts in a Phase I clinical trial (NCT03585322).

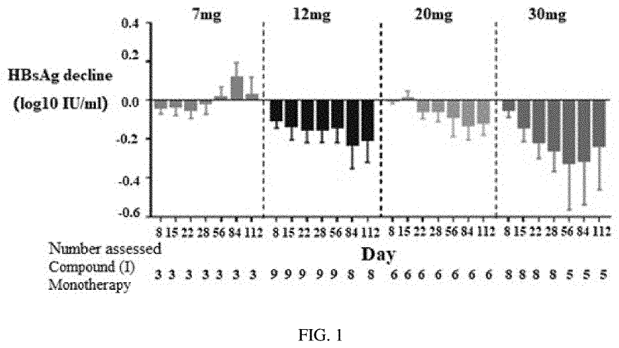

### DETAILED DESCRIPTION OF THE INVENTION

The disclosure provides improved methods for treating HBV in patients in need thereof by administering Compound (I) and/or a pharmaceutically acceptable salt thereof. Specifically, the disclosure provides safe and effective dosing regimens of Compound (I) that can be used long-term treatment.

As used herein, “Compound (I)” refers to a compound having a chemical name (5S ,5′S ,8S ,8′S,10aR,10a′R)-3,3′-(1,3-phenylenedisulfonyl)bis(N-benzhydryl-5-((S)-2-(methylamino)propanamido)-6-oxodecahydropyrrolo[1,2-a][1,5]diazocine-8-carboxamide), which has the following structure:

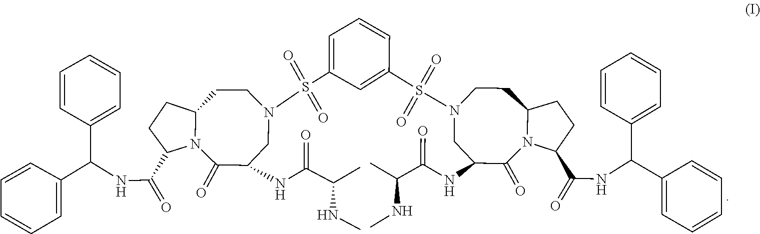

Compound (I) is a bivalent small molecule Smac mimetic that antagonizes the IAPs (inhibitor of apoptosis proteins). In some studies, SMAC mimetic can quickly promote the clearance of HBsAg and HBV DNA in chronic HBV infection mice model by antagonistic IAPs. This will be a major breakthrough in HBV curing treatment and a completely new treatment strategy. In preclinical studies, it is demonstrated that Compound (I) inhibits IAPs in vivo and in vitro, and it can clear chronic HBV infections in different models. It shows potential application of Compound (I) in HBV infection treatment with primary investigation on its mechanism of action in this disease area.

The preparation of Compound (I) is described in Example 24 of WO2014/031487. The use of Compound (I) to treat hepatitis virus-related diseases or disorders is also disclosed in WO2019/101057. Both references are expressly incorporated herein in their entireties by reference.

As used herein, the term “pharmaceutically acceptable salt” refers to a non-toxic salt form of a compound of this disclosure. Pharmaceutically acceptable salts of Compound (I) include those derived from suitable inorganic and organic acids and bases. Pharmaceutically acceptable salts are well known in the art. Suitable pharmaceutically acceptable salts are, e.g., those disclosed in Berge, S. M., et al. J. Pharma. Sci. 66: 1-19 (1977). Non-limiting examples of pharmaceutically acceptable salts disclosed in that article include: acetate; benzenesulfonate; benzoate; bicarbonate; bitartrate; bromide; calcium edetate; camsylate; carbonate; chloride; citrate; dihydrochloride; edetate; edisylate; estolate; esylate; fumarate; gluceptate; gluconate; glutamate; glycollylarsanilate; hexylresorcinate; hydrabamine; hydrobromide; hydrochloride; hydroxynaphthoate; iodide; isethionate; lactate; lactobionate; malate; maleate; mandelate; mesylate; methylbromide; methylnitrate; methylsulfate; mucate; napsylate; nitrate; pamoate (embonate); pantothenate; phosphate/diphosphate; polygalacturonate; salicylate; stearate; subacetate; succinate; sulfate; tannate; tartrate; teociate; triethiodide; benzathine; chloroprocaine; choline; diethanolamine; ethylenediamine; meglumine; procaine; aluminum; calcium; lithium; magnesium; potassium; sodium; and zinc.

Non-limiting examples of pharmaceutically acceptable salts derived from appropriate acids include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Additional non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Non-limiting examples of pharmaceutically acceptable salts derived from appropriate bases include alkali metal, alkaline earth metal, ammonium, and N+(C1-4 alkyl)4 salts. This disclosure also envisions the quaternization of any basic nitrogen-containing groups of the compounds disclosed herein. Non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other non-limiting examples of pharmaceutically acceptable salts include besylate and glucosamine salts.

As used herein, the term “hepatitis B” or “HB”, refers to an infectious disease caused by the hepatitis B virus (HBV) that affects the liver.

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

HBsAg is the serological hallmark of HBV infection. After an acute exposure to HBV, HBsAg appears in serum within 1 to 10 weeks. Persistence of this marker for more than 6 months implies chronic HBV infection. Several studies have reported the association between transcription activity of cccDNA in the liver and serum HBsAg levels. Differences in the serum HBsAg levels during the different phases of infection indicate the distribution of cccDNA during the respective phases of the disease. The serum HBsAg titers are higher in patients with HBeAg-positive CHB than in HBeAg-negative CHB. Monitoring of quantitative HBsAg levels predicts treatment response to interferon and disease progression in HBeAg negative CHB patients with normal serum alanine aminotransferase levels.

In some embodiments, the HBsAg level in a patient with HB (or HBV infection) is reduced over ≥0.2 log, after the patient is treated with Compound (I) in a dosage as described herein. In some embodiments, the HBsAg level in a patient with HB (or HBV infection) is reduced over ≥0.3 log, after the patient is treated with Compound (I) in a dosage as described herein. In some embodiments, the HBsAg level in a patient with HB (or HBV infection) is reduced over ≥0.4 log, after the patient is treated with Compound (I) in a dosage as described herein. In some embodiments, the HBsAg level in a patient with HB (or HBV infection) is reduced over ≥0.5 log, after the patient is treated with Compound (I) in a dosage as described herein.

In some embodiments, the HBV DNA level in a patient with HB (or HBV infection) is reduced over 0.01 log10 IU/ml, after the patient is treated with Compound (I) in a dosage as described herein. In some embodiments, the HBV DNA level in a patient with HB (or HBV infection) is reduced over 0.05 log10 IU/ml, after the patient is treated with Compound (I) in a dosage as described herein. In some embodiments, the HBV DNA level in a patient with HB (or HBV infection) is reduced over 0.1 log10 IU/ml, after the patient is treated with Compound (I) in a dosage as described herein. In some embodiments, the HBV DNA level in a patient with HB (or HBV infection) is reduced over 0.5 log10 IU/ml, after the patient is treated with Compound (I) in a dosage as described herein. In some embodiments, the HBV DNA level in a patient with HB (or HBV infection) is reduced over 1 log10 IU/ml, after the patient is treated with Compound (I) in a dosage as described herein. In some embodiments, the HBV DNA level in a patient with HB (or HBV infection) is reduced over 1.5 log10 IU/ml, after the patient is treated with Compound (I) in a dosage as described herein. In some embodiments, the HBV DNA level in a patient with HB (or HBV infection) is reduced over 2.0 log10 IU/ml, after the patient is treated with Compound (I) in a dosage as described herein. In some embodiments, the HBV DNA level in a patient with HB (or HBV infection) is reduced over 2 log10 IU/ml, after the patient is treated with Compound (I) in a dosage as described herein.

As used herein, “effective amount” of a compound disclosed herein refers to an amount of the compound that will elicit a biological or medical response in a subject, e.g., reduce or inhibit enzyme or protein activity, ameliorate symptoms, alleviate conditions, or slow or delay disease progression.

As used herein, the term “inhibit,” “inhibition,” or “inhibiting” refers to the reduction or suppression of a given condition, symptom, disorder, or disease, or a significant decrease in the baseline activity of a biological activity or process.

As used herein, the term “patient” or “subject” refers to an organism to be treated by the methods of the disclosure. Non-limiting example organisms include mammals, e.g., murines, simians, equines, bovines, porcines, canines, felines, and the like. In some embodiments, the organism is a human. In some embodiments, the patient population comprises patients having HBV with moderate-to-severe symptoms that cannot be adequately controlled with approved symptom-directed therapies.

As used herein, the term “treat,” “treating,” or “treatment,” when used in connection with a disorder or condition, includes any effect, e.g., lessening, reducing, modulating, ameliorating, and/or eliminating, that results in the improvement of the disorder or condition. Improvements in or lessening the severity of any symptom of the disorder or condition can be readily assessed according to standard methods and techniques known in the art.

As used herein, the terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The disclosure provides a method of treating HB (e.g., chronic hepatitis B) or HBV infection comprising administering to a patient in need thereof an amount of 7 mg to 45 mg of Compound (I) and/or a pharmaceutically acceptable salt thereof once a week. In some embodiments, the amount is 7 mg, 10 mg, 12 mg, 15 mg, 20 mg, 25 mg, 30 mg, 35 mg, 40 mg, or 45 mg once a week. In some embodiments, the amount is 7 mg once a week. In some embodiments, the amount is 12 mg once a week. In some embodiments, the amount is 20 mg once a week. In some embodiments, the amount is 30 mg once a week. In some embodiments, the amount is 45 mg once a week.

In certain embodiments, Compound (I) is administered in an amount of about 0.005 mg/day to about 5000 mg/day, such as about 0.005, 0.05, 0.5, 5, 9, 10, 20, 30, 40, 50, 60, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day. In certain embodiments, Compound (I) is administrated in an amount of about 10 mg/week to about 200 mg/week, or about 20 mg/week to about 100 mg/week, or about 20 mg/week to about 80 mg/week, such as 10, 15, 20, 25, 30, 35, 40, 50, 55, 60, 65, 70, 75, 80, 85, 90, 95, 100, 110, 120, 130, 140, 150, 160, 170,180, 190, or 200 mg/week.

In certain embodiments, Compound (I) is administered in an amount of about 1 ng/kg to about 200 mg/kg, about 1 μg/kg to about 100 mg/kg, or about 1 mg/kg to about 50 mg/kg per unit dose, such as administered in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 1.5 mg/kg, about 2.5mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, and about 200 mg/kg per unit dose, and one or more (e.g., 1, 2, 3, 4, 5, 6, 7, 8, 9 or 10) unit doses are administered every day, every 2 days, every 3 days, every 4 days, every 5 days, every 6 days, or every week.

In certain embodiments, Compound (I) is administered 1, 2, 3, 4, 5, 6, or 7 times every week. In some embodiments, Compound (I) is administered continuously for at least 1 week, at least 2 weeks, at least 3 weeks, at least 4 weeks, at least 5 weeks, at least 6 weeks, at least 7 weeks, or at least 8 weeks.

In certain embodiments, Compound (I) is administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

In certain embodiments, Compound (I) is administered for one or more (e.g., 1, 2, 3, 4, 5, 6, 7, 8, 9 or 10) courses of treatment, wherein each course of treatment lasts for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days or at least 50 days, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks; wherein for each course of treatment, administration is performed 1, 2, 3, 4, 5, 6, 7, 8, 9 or 10 times; and the interval between every two courses of treatment is 0, 1, 2, 3, 4, 5, 6, 7, 8, 9, 10 days, 2 weeks, 3 weeks, 4 weeks, 1 month or 2 months.

In some embodiments, Compound (I) or a pharmaceutically acceptable salt thereof is administered together with an amount of 0.2 mg to 1 mg (e.g., 0.5 mg or 1 mg) of entecavir. In some embodiments, Compound (I) and entecavir are administered concurrently. In some embodiments, Compound (I) and entecavir are administered sequentially.

In some embodiments, in the methods disclosed herein, Compound (I) or a pharmaceutically acceptable salt thereof is administered once a week. In some embodiments, in the methods disclosed herein, entecavir is administered once a day.

In some embodiments, the method disclosed herein comprises at least one 21-day treatment cycle, wherein Compound (I) or a pharmaceutically acceptable salt thereof is administrated on days 1, 8, and 15 of the consecutive 3-weeks of the treatment cycle. In some embodiments, the method disclosed herein comprises at least one 28-day treatment cycle, wherein Compound (I) or a pharmaceutically acceptable salt thereof is administrated on days 1, 8, 15, and 22 of the consecutive 4-weeks of the treatment cycle.

Compound (I) and/or pharmaceutically acceptable salts thereof described herein are useful as an active pharmaceutical ingredients (API) as well as materials for preparing pharmaceutical compositions that incorporate one or more pharmaceutically acceptable excipients and is suitable for administration to human subjects.

In some embodiments, the disclosure provides a pharmaceutical composition comprising Compound (I) and/or a pharmaceutically acceptable salt thereof and at least one additional pharmaceutically acceptable excipient. The term “pharmaceutically acceptable excipient,” as used herein, refers to a pharmaceutically acceptable material, composition, and/or vehicle, such as a liquid or solid filler, diluent, excipient, solvent, or encapsulating material. Each excipient must be “pharmaceutically acceptable” in the sense of being compatible with the subject composition and its components and not injurious to the patient. Except insofar as any conventional pharmaceutically acceptable excipient is incompatible with Compound (I) and/or pharmaceutically acceptable salts thereof, such as by producing any undesirable biological effect or otherwise interacting in a deleterious manner with any other component(s) of the pharmaceutically acceptable composition, its use is contemplated to be within the scope of this disclosure.

Some non-limiting examples of materials which may serve as pharmaceutically acceptable excipients include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, also disclose additional non-limiting examples of pharmaceutically acceptable excipients, as well as known techniques for preparing and using the same.

Pharmaceutical compositions disclosed herein may be administered orally, intravenously, by inhalation spray, topically, rectally, nasally, buccally, vaginally, or via an implanted reservoir. In some embodiments, the compositions of the disclosure are administered orally or intravenously. Sterile injectable forms of the pharmaceutical compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3-butanediol. Non-limiting examples of acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween, Spans, and other emulsifying agents or bioavailability enhancers which are commonly used in the manufacture of pharmaceutically acceptable solid, liquid, or other dosage forms may also be used for the purposes of formulation.

Pharmaceutical compositions disclosed herein may also be orally administered in any orally acceptable dosage form including, but not limited to, capsules, tablets, aqueous suspensions, or solutions. When aqueous suspensions are required for oral use, the active ingredient is typically combined with emulsifying and suspending agents. If desired, certain sweetening, flavoring, or coloring agents may also be added. In some embodiments, the pharmaceutical composition comprising Compound (I) and/or a pharmaceutically acceptable salt thereof is a tablet prepared using methods known in the art. In some embodiments, the tablet is an immediate release tablet for oral administration. In some embodiments, Compound (I) and/or a pharmaceutically acceptable salt thereof is blended with pharmacopeial excipients to form an immediate release tablet. In some embodiments, the excipients comprising the tablet are microcrystalline cellulose, copovidone, croscarmellose sodium, and magnesium sterate. In some embodiments, the formulation blend is roller compacted, compressed into round tablets, and aesthetically film coated.

### EXAMPLES

The following examples are intended to be illustrative and are not meant in any way to limit the scope of the disclosure.

### Example 1

Methods: This study (NCT number: NCT03585322) is a multi-center, single-agent, open-label, Phase I dose-escalation study. Chronic hepatitis B (CHB) patients without receiving anti-HBV treatment within 6 months before screening were recruited and administrated with Compound (I) weekly via intravenous infusion (IV) in 4 escalated dosage (7 mg, 12 mg, 20 mg and 30 mg) for 4 weeks followed with 12 weeks follow up. The primary objective of this study is to assess the safety, with secondary outcome measures focused on pharmacokinetics (PK) and Pharmacodynamics (PD) of Compound (I) as monotherapy in CHB patients.

Results: A total of 25 CHB patients completed 4 doses of Compound (I) while one patient received single dose of 30 mg and observed for 4 weeks only. The most common (>10%) drug-related adverse events were ALT (Alamine aminotransferase) increase (26%), AST (Aspartate aminotransferase) increase (22%), total bile acids increase (15%), infusion site pain (15%) or infusion site reaction (15%) respectively. Four patients had grade 3 ALT or AST elevation (5-15× ULN). A grade 2 facial nerve disorder occurred in 1 patient in 30 mg cohort and recovered within 4 weeks. PK analysis indicated a dose proportional increase in plasma exposure and no accumulation after multiple dosing. Reduction of HBsAg was observed in 17 of 26 (65%) patients with range from 0.04 to 0.79 log10 IU/ml on week 4. Furthermore, reduction of HBsAg at ≥0.2 log and ≥0.5 log was achieved with 46% ( 12/26) and 19% ( 5/26) patients respectively. Notably, 11/23 (48%) patients achieved prolonged HBsAg reduction during the post-treatment follow-up, with range from 0.08 to 1.22 log10 IU/ml. Moreover, decline of HBV DNA was found in 23 of 26 (89%) patients with range from 0.01 to 2.61 log10 IU/ml, and ALT normalization was found in 10/22 (46%) patients.

Conclusions: In this phase I dose-escalating study of Compound (I) in CHB, Compound (I) was safe and well tolerated following single ascending doses from 7 mg to 30 mg. Reductions of HBsAg and HBV DNA levels were observed with only 4 weekly dosing of monotherapy, indicating the Compound (I) had the antiviral potency for CHB therapy.

TABLE 1
Baseline Characteristics, Safety, PD and other findings

| | | 7 mg | | 12 mg | | 20 mg | | 30 mg | |
|---|---|---|---|---|---|---|---|---|---|
| | Mean(SD) | Compound (I) | | Compound (I) | | Compound (I) | | Compound (I) | |
| | except where noted | (n = 3) | | (n = 9) | | (N = 6) | | (N = 6) | |
| Baseline | Male N (%) | 3 | (100.0%) | 6 | (66.7%) | 2 | (33.3%) | 4 | (66.7%) |
| Demographics and | Asian N (%) | 3 | (100.0%) | 9 | (100.0%) | 6 | (100.0%) | 6 | (100.0%) |
| Disease | HBeAg positive N (%) | 2 | (66.7%) | 5 | (55.6%) | 5 | (83.3%) | 4 | (66.7%) |
| Characteristics | Baseline Mean HBsAg Log | 3.803 | (0.9604) | 3.662 | (0.7278) | 4.338 | (0.8612) | 4.012 | (0.3671) |
| | IU/ml | | | | | | | | |
| | Fibro test Score (kPa) | 5.90 | (0.600) | 6.92 | (1.974) | 5.60 | (1.375) | 7.78 | (3.024) |
| | Mean(SD) | | | | | | | | |
| Safety | Any Drug-Related TEAE N (%) | 2 | (66.7%) | 3 | (33.3%) | 6 | (100.0%) | 5 | (83.3%) |
| | SAE N (%) | 0 | (0%) | 0 | (0%) | 0 | (0%) | 1 | (16.7%) |
| Pharmacodynamics | HBsAg (IU/ML) | −0.01 | (0.10) | −0.18 | (0.20) | −0.05 | (0.14) | −0.27 | (0.37) |
| (PD) | Changes from baseline to end | | | | | | | | |
| | of treatment(D28) | | | | | | | | |
| | HBV DNA (IU/ML) | −0.18 | (0.27) | −0.72 | (0.69) | −0.60 | (1.01) | −0.55 | (0.44) |
| | Changes from baseline to end | | | | | | | | |
| | of treatment (D28) | | | | | | | | |
| Other finding | Fibro Test Score (kPa) | 0.27 | (0.586) | −1.80 | (2.615) | −0.33 | (1.231) | −4.05 | (0.354) |

| | | | | |
|---|---|---|---|---|
| Changes from baseline to end | N = 3 | N = 3 | N = 6 | N = 2 |
| of follow-up | | | | |

### Example 2. A phase II Clinical Study of Compound (I) in Combination With Entecavir in Patients With Chronic Hepatitis B

This study is a multi-center, open-label, phase II clinical study in patients with chronic hepatitis B. The primary objective of the study is to assess the safety, tolerability, pharmacokinetic characteristics and preliminary anti-HBV efficacy of Compound (I) at different doses in combination with entecavir. The second objective is to determine the recommended clinical dose of Compound (I) in combination with entecavir to treat chronic hepatitis B.

The study will be conducted in two phases. In the first phase, patients are administered with Compound (I) in 3 escalated dose (12 mg, 20 mg, and 30 mg) in combination with a fixed-dose entecavir tablet (0.5 mg/day) for 4 weeks, followed by entecavir monotherapy for 8 weeks for a total of 12 weeks. A standard “3+3” escalation scheme is used to determine the maximum tolerated dose (MTD) of Compound (I) by assessing the dose-limiting toxicity (DLT) of Compound (I) in combination with entecavir. The DLT is based on National Cancer Institute Common Terminology Criteria for Adverse Events (NCI CTCAE) v5.0 classification. 3 to 6 patients are included in each dose group. The interval of the first administration between the first patient and the other patients in the same dose group shall be no less than 24 hours (the observation period for the acute allergic reaction of the drug).

The study begins at the lowest dose level (12 mg) of Compound (I) in combination of entecavir tablets (0.5 mg/day) for 4 weeks. If no DLT is observed within 28 days in the first 3 patients, the dose of Compound (I) will be increased to next dose level (20 mg and 30 mg). If ≥1 patient develops DLT, or encounters Compound (I) related or possibly Compound (I) related serious adverse events, the patient will discontinue the study immediately. The sponsors and the investigators will evaluate the safety data of the whole dose group and will evaluate whether serious damage has been incurred to the patient's health, in order to determine whether to continue the study to the patient. In the meantime, three more patients will be added to same dose group. If no new DLT is observed after addition of the new patients, the next escalated dose will be studied. If ≥2 patients develop DLT at any dose level, dose escalation will cease and the previous lower dose level will be declared as maximum tolerated dose (MTD) (if this occurs in the 12 mg group, the study will be terminated).

After the safety assessment of all patients in each dose group is completed on day 28, the sponsors and the investigators will evaluate the safety data. With the consent of the sponsors and the investigators, the next dose group study will be initiated. After the safety assessment of all patients in the MTD group is completed on day 28, based on the safety, pharmacokinetics and preliminary efficacy results, the second phase of the study will be initiated with the consent of the sponsors and the investigators. All the patients in the first phase study will continue entecavir monotherapy and be followed up for 8 weeks, with an overall observation period of 12 weeks.

The second phase is a randomized open-label study, which compares the preliminary anti-HBV efficacy of Compound (I) at different doses in combination with entecavir with the efficacy of entecavir monotherapy. For now, the design of the study is based on the MTD being 30 mg for Compound (I) combined with entecavir tablets. Chronic hepatitis B patients will be randomly divided into four study groups at 1:1:1:1, including the active drug control group (entecavir monotherapy for 24 weeks) and the three combination treatment groups (treated with Compound (I) 12 mg, 20 mg, and 30 mg in combination with entecavir tablets for 12 weeks, and then entecavir monotherapy for another 12 weeks). The total course of treatment in each group is 24 weeks.

Patients in the combination treatment groups will be administered Compound (I) at a prescribed dose every 7 days via intravenous infusion for a total of 12 weeks. All the patients will be administered 1 entecavir tablet (0.5 mg) at a fixed time period daily in fasting condition for 24 weeks. During the study, no other chronic hepatitis B drugs, including antiviral, immunomodulatory, antifibrotic, and symptomatic treatment not caused by adverse events will be used.

1. A method of treating hepatitis B (HB) comprising administering to a patient in need thereof an amount of 7 mg to 45 mg of Compound (I) or a pharmaceutically acceptable salt thereof once a week, wherein Compound (I) is represented by the structural formula below:

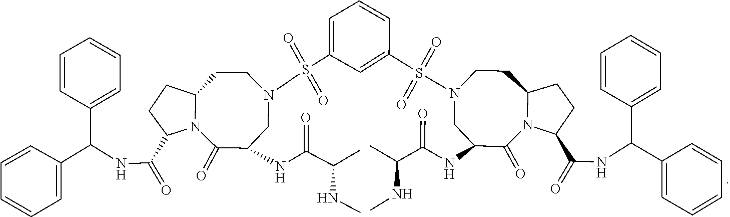

2. The method of claim 1, wherein the amount is 7 mg.

3. The method of claim 1, wherein the amount is 12 mg.

4. The method of claim 1, wherein the amount is 20 mg.

5. The method of claim 1, wherein the amount is 30 mg.

6. The method of claim 1, wherein Compound (I) is administered intravenously.

7. A method of treating hepatitis B, comprising administering to a subject in need thereof:
a) an amount of 7 mg to 45 mg of Compound (I) represented by the structural formula below: 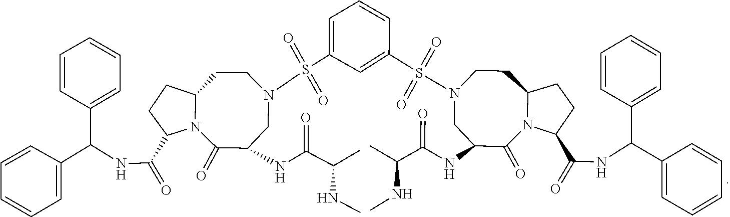
or a pharmaceutically acceptable salt thereof; and
b) an amount of 0.2 mg to 1 mg of entecavir.

8. The method of claim 7, wherein Compound (I) is administered once a week.

9. The method of claim 8, wherein entecavir is administered once a day.

10. The method of claim 9, wherein the method comprises at least one 28-day treatment cycle, wherein Compound (I) is administrated on days 1, 8, 15, and 22 of the consecutive 4-weeks of the treatment cycle.

11. The method of claim 10, wherein the effective amount of Compound (I) is 12 mg.

12. The method of claim 10, wherein the effective amount of Compound (I) is 20 mg.

13. The method of claim 10, wherein the effective amount of Compound (I) is 30 mg.

14. The method of claim 7, wherein the effective amount of entecavir is 0.5 mg.

15. The method of claim 7, wherein the effective amount of entecavir is 1 mg.

16. The method of claim 7, wherein Compound (I) is administered via an intravenous infusion.

17. The method of claim 7, wherein Compound (I) and entecavir are administered concurrently.

18. The method of claim 7, wherein Compound (I) and entecavir are administered sequentially.

19. The method of claim 7, wherein hepatitis B is chronic hepatitis B.

20. The method of claim 7, wherein Compound (I) is a free base.
